(19) United States
(12) Patent Application Publication
(10) Pub. No.: US 2020/0237388 A1
(43) Pub. Date: Jul. 30, 2020

(54) METHODS AND APPARATUS FOR DELIVERING A STIMULUS TO AN OCCLUSIVE IMPLANT

(71) Applicant: Contraline, Inc., Charlottesville, VA (US)

(72) Inventors: Kevin Eisenfrats, Charlottesville, VA (US); Greg Grover, Charlottesville, VA (US); Eric Moran, White Post, VA (US)

(21) Appl. No.: 16/753,302

(22) PCT Filed: Oct. 2, 2018
(86) PCT No.: PCT/US18/53853

§ 371 Date: Apr. 2, 2020

Related U.S. Application Data
(60) Provisional application No. 62/566,592, filed on Oct. 2, 2017.

(51) Int. Cl. A61B 17/22; A61N 5/06; A61F 6/22

(52) U.S. Cl. CPC A61B 17/22; A61N 5/0601; A61B 17/1219; A61B 2218/002; A61F 6/22

(57) ABSTRACT

An apparatus, system or device and methods for applying one or more stimuli to an implantation, occlusive device, or embolization are described. The implantation can include a polymer or polymer composition such as a hydrogel and can be used for sterilizing a subject in need of contraception by occluding the vas deferens, fallopian tube(s), or uterus, but can also be used to occlude, in whole or part, any other body ducts or organs. The one or more stimuli can modify the occlusion so that is removed from the body lumen in situ, there by reversing the contraception or other effects of the occlusion.

### CROSS-REFERENCE TO RELATED APPLICATIONS

This application relies on the disclosure of and claims priority to and the benefit of the filing date of U.S. Provisional Application No. 62/566,592 filed Oct. 2, 2017, the disclosure of which is hereby incorporated by reference in its entirety.

### BACKGROUND OF THE INVENTION

### Field of the Invention

The present invention relates to the field of medical devices and more particularly to medical devices and methods for treating implants in a body in situ, for example, occlusions.

### Description of Related Art

Implants are artificial objects, materials, or tissues that are inserted, injected, fixed, or implanted into a body, such as by surgery. Implants for occluding vessels such as hydrogel implants are known; however, there is a lack of means for removing hydrogel implants safely and effectively. Hydrogels are highly hydrated polymer chains or networks that are able to absorb significant volumes of water and can have tunable mechanical properties. Other definitions of a hydrogel are: 1) a water-swollen, and cross-linked polymeric network produced by the simple reaction of one or more monomers; and 2) a polymeric material that exhibits the ability to swell and retain a significant fraction of water within its structure, but will not dissolve in water. (see Enas M. Ahmed, Hydrogel: Preparation, characterization, and applications: A review, Journal of Advanced Research, Volume 6, Issue 2, 2015, Pages 105-121). Examples of related efforts in this area include those described in U.S. Pat. Nos. 4,887,605; 5,248,311; 5,437,660; 6,461,569; 8,523,848; 8,096,478; 4,273,109; 5,607,419; 6,379,373; 6,660,247; 6,723,090; 6,802,838; 9,180,196; U.S. Patent Application Publication Nos. US20100068153A1, US20120149781A1, US20120192872A1, US 20160153999 A1, US 20120228520A1, and International Patent Application Publication No. WO2015168090A1, as well as in WO/2017/083753, WO/2018/139369, US20170136144, and US20170136143 (each of which are incorporated by reference herein in their entirety).

There is a large unmet need in the field of medicine for an apparatus that may apply a stimulus to a hydrogel implant in situ, such as for reversing the hydrogel or implant. There is increasing interest and application for stimuli-responsive implants (see US20180185096A1). One such example of a stimuli-responsive implant is a photoreversible hydrogel. These hydrogels are chemically designed to be responsive to certain wavelengths and intensities of light. Photoreversible hydrogels may be used for short or long-lasting occlusion of bodily ducts, for instance, or for drug delivery applications. Upon administering light to the hydrogel, the implant may undergo a chemical response, such that the gel is broken down, deteriorated, removed, reversed, or dissolved such that the occlusion is no longer present or the occlusive effect is diminished or negated. In drug-delivery applications, the light may be used to release drugs from the hydrogel implant.

Existing apparatuses for introducing stimuli, such as light, into the body are designed to apply stimuli to the body's own cells, tissues, or byproducts such as for lithotripsy, tissue ablation purposes, or inducing lesion formation rather than for artificial implants such as removing stimulus-responsive hydrogels. Lasers for stone lithotripsy, for example, are used for removing stones from kidneys, ureters, bladders, urethras, gall bladders, and more. The apparatus uses lasers to fragment stones into tiny pieces, where the pieces are then removed. Occasionally, the stone will be fragmented into very small pieces, too small to be basketed, and as such, the physician usually allows those pieces to clear themselves over time. Most lithotripsy apparatuses employ Holmium yttrium-aluminum-garnet (Ho:YAG) laser, which has a wavelength of 2.1 μm (2,100 nm) and thus, falls in the infrared spectra. These lasers have similar qualities of carbon dioxide and Nd:Yag lasers (which also fall in the infrared spectra), but Ho:YAG lasers have ablative and coagulation effects. These high-wavelength lasers also induce a temperature rise in the laser-affected zone, which helps to break down the stone or ablate tissue, but would present problems if used on normal tissues.

Hydrogel implants are often implanted into areas where the patient and/or physician desire the area to remain undamaged once the implant is removed. For example, a laser has been shown to be used for irradiating the vas deferens for non-invasive vasectomy, a permanent procedure (U.S. Pat. No. 8,523,848B2). However, this laser would not be desired for applying a light stimulus to a stimulus-responsive hydrogel within the lumen of the vas deferens for reversible contraception, as unintended sterilization may occur.

### SUMMARY OF THE INVENTION

Embodiments of the present invention are directed to an apparatus and methods for applying stimuli to an occlusion, such as but not limited to an implant, occlusive device, or embolization of a body part, such as a body lumen. The occlusion can include a polymer or polymer composition such as a hydrogel or insoluble polymer network and can be used for providing contraception to a subject by occluding the vas deferens, fallopian tube(s), or uterus, but can also be used to occlude any other body part, such as ducts, tissues, interstitial spaces, or organs such as for drug delivery or bulking purposes, for example. Embodiments of the present invention can be used to change the properties of the implant such that, for example, the occlusion is reversed, drugs are released from the implant, or the implant's swelling, mesh/pore size, charge, and/or solubility can be increased or reduced. Embodiments can also be used to apply a variety of stimuli to an implantation in any other body duct, tissue, interstitial space, or organ.

### BRIEF DESCRIPTION OF THE DRAWINGS

The accompanying drawings illustrate certain aspects of embodiments of the present invention, and should not be used to limit the invention. Together with the written description the drawings serve to explain certain principles of the invention.

FIG. 1 is a schematic diagram of a system and apparatus according to an embodiment, which can be used with methods of the invention.

FIG. 2 is a schematic diagram of a system and apparatus according to an embodiment, which can be used with methods of the invention.

FIG. 3 is a schematic diagram showing a device as well as a cross-section of the device according to an embodiment of the invention.

FIG. 4 is a schematic diagram showing delivery of a stimulus to an occlusion in the lumen of the vas deferens through a device according to an embodiment of the invention.

FIG. 5 is a schematic diagram showing delivery of a stimulus to an occlusion in the lumen of an oviduct through a device according to an embodiment of the invention.

FIG. 6A is a schematic diagram showing administration of an occlusive device, such as an occlusive hydrogel, into a lumen according to an embodiment of the invention.

FIG. 6B is a schematic diagram showing a contraceptive occlusive hydrogel implant in the vas deferens according to an embodiment of the invention.

FIG. 6C is a schematic diagram showing introduction of a stimulus into a lumen to degrade and/or flush the occlusive hydrogel according to an embodiment of the invention.

FIG. 7 is a bar graph which shows the chemical transformation of a photolabile molecule in solution after short exposure to light using the device according to embodiments of the invention.

FIG. 8 is a bar graph which shows the change in the rheological properties of a hydrogel implant after being exposed to a stimulus according to an embodiment of the invention.

### DETAILED DESCRIPTION OF VARIOUS EMBODIMENTS OF THE INVENTION

Reference will now be made in detail to various exemplary embodiments of the invention. It is to be understood that the following discussion of exemplary embodiments is not intended as a limitation on the invention. Rather, the following discussion is provided to give the reader a more detailed understanding of certain aspects and features of the invention.

In embodiments, the present invention describes an apparatus that is able to deliver a stimulus or stimuli to change an implant disposed in a body, such as in a vessel lumen, body duct, tissue, interstitial space, or organ. Some of the embodiments described below focus on delivery of electromagnetic radiation (EMR) and the impact of the delivered EMR on implants, however, the ability to deliver other stimuli is included as well.

In embodiments, the implant is a polymeric medical device, and an apparatus delivers a stimulus to change the properties of the implant such that it disintegrates, de-precipitates, dislodges, dissolves, or releases drugs. Examples of reversal mechanisms encompassed by stimuli delivered by the apparatus of the invention can include, but are not limited to, photodegradation (e.g. ultraviolet, visible, monochromatic, or infrared exposure), ultrasound, mechanical, electrical, physical, vibrational, magnetic, pH-based, temperature-based, ionic, retro “Click” chemistry, and/or enzymatic degradation, and any combination thereof. In embodiments, the stimulus is electromagnetic radiation, energy, sound waves, heat, vibrations, aqueous solutions (neutral, basic, or acidic), organic solvents, aqueous-organic mixtures, enzyme(s), protein(s), peptide(s), small organic molecules (<500 g/mole), large organic molecules (> or =500 g/mole), nanoparticles, microparticles, quantum dots, carbon-based materials, and/or any combination thereof.

In embodiments, the medical device, such as a polymeric medical device, can be in the form of an implant, hydrogel, gel, mesh, stent, film, embolization, composition, or device (herein referred to interchangeably as an implant, hydrogel, gel, mesh, embolization, composition, device, occlusive device, occlusive composition, occlusive substance, or any other applicable definition of gel, mesh, composition, device, formulation, or other object or article). In the context of this disclosure, the terms occlusion, occlusive, occlude, occluding and the like refer to the act of occupying space and include but are not limited to blocking, obstructing, disrupting, interfering with, or preventing, in whole or part, movement of a substance from one area to another. In embodiments, the medical device, such as polymer gel, is implanted into the vas deferens or fallopian tubes for male and female contraception, respectively, and can have the function of blocking or otherwise interfering with sperm or the oocyte from traveling within, through or into the relevant tube(s), duct(s), and/or organ(s), thus causing temporary or permanent infertility; preferably, temporary infertility because the implant can be reversed.

In embodiments, the apparatus, system, and methods of the invention can be used to change the properties of the implant within a bodily duct, lumen, vessel, tissue, interstitial space, or organ. The implant can be used to occlude the reproductive duct(s) of a mammal (e.g. vas deferens or fallopian tube) to cause contraception or sterilization. One result of the change in properties of the implant after exposure to the stimulus is that the implant is no longer able to occlude the duct or vessel. In the case of contraception, this change would restore fertility.

In one embodiment, the invention is used to form an implant, such as to cure or polymerize a hydrogel. In one aspect, the invention delivers a stimulus to enable formation of the occlusive composition.

The implant can comprise one or more of natural or synthetic monomers, polymers or copolymers, biocompatible monomers, polymers or copolymers, such as polystyrene, neoprene, polyetherether 10 ketone (PEEK), carbon reinforced PEEK, polyphenylene, polyetherketoneketone (PEKK), polyaryletherketone (PAEK), polyphenylsulphone, polysulphone, polyurethane, polyethylene, low-density polyethylene (LDPE), linear low-density polyethylene (LLDPE), high-density polyethylene (HDPE), polypropylene, polyetherketoneetherketoneketone (PEKEKK), nylon, fluoropolymers such as polytetrafluoroethylene (PTFE or TEFLON®), TEFLON® TFE (tetrafluoroethylene), polyethylene terephthalate (PET or PETE), TEFLON® FEP (fluorinated ethylene propylene), TEFLON® PFA (perfluoroalkoxy alkane), and/or polymethylpentene (PMP) styrene maleic anhydride, styrene maleic acid (SMA), polyurethane, silicone, polymethyl methacrylate, polyacrylonitrile, poly (carbonate-urethane), poly (vinylacetate), nitrocellulose, cellulose acetate, urethane, urethane/carbonate, polylactic acid, polyacrylamide (PAAM), poly (N-isopropylacrylamine) (PNIPAM), poly (vinylmethylether), poly (ethylene oxide), poly (ethyl (hydroxyethyl) cellulose), poly(2-ethyl oxazoline), polylactide (PLA), polyglycolide (PGA), poly(lactide-co-glycolide) PLGA, poly(e-caprolactone), polydiaoxanone, polyanhydride, trimethylene carbonate, poly(β-hydroxybutyrate), poly(g-ethyl glutamate), poly(DTH-iminocarbonate), poly(bisphenol A iminocarbonate), poly(orthoester) (POE), polycyanoacrylate (PCA), polyphosphazene, polyethyleneoxide (PEO), polyethylene glycol (PEG) or any of its derivatives, polyacrylacid (PAA), polyacrylonitrile (PAN), polyvinylacrylate (PVA), polyvinylpyrrolidone (PVP), polyglycolic lactic acid (PGLA), poly(2-hydroxypropyl methacrylamide) (pHPMAm), poly(vinyl alcohol) (PVOH), PEG diacrylate (PEGDA), poly(hydroxyethyl methacrylate) (pHEMA), N-i sopropylacrylamide (NIPA), polyoxazoline (POx), poly(vinyl alcohol) poly(acrylic acid) (PVOH-PAA), collagen, silk, fibrin, gelatin, hyaluron, cellulose, chitin, dextran, casein, albumin, ovalbumin, heparin sulfate, starch, agar, heparin, alginate, fibronectin, fibrin, keratin, pectin, elastin, ethylene vinyl acetate, ethylene vinyl alcohol (EVOH), polyethylene oxide, PLLA or P1LA (poly(L-lactide) or poly(L-lactic acid)), poly(D,L-lactic acid), poly(D,L-lactide), poly dim ethyl siloxane or dimethicone (PDMS), poly(isopropyl acrylate) (PIPA), polyethylene vinyl acetate (PEVA), PEG styrene, polytetrafluoroethylene RFE such as TEFLON® RFE or KRYTOX® RFE, fluorinated polyethylene (FLPE or NALGENE®), methyl palmitate, temperature responsive polymers such as poly(N-i sopropylacrylamide) (NIPA), polycarbonate, polyethersulfone, polycaprolactone, polymethyl methacrylate, polyisobutylene, nitrocellulose, medical grade silicone, cellulose acetate, cellulose acetate butyrate, polyacrylonitrile, poly(lactide-co-caprolactone (PLCL), and/or chitosan.

In one embodiment, the implant is a hydrogel comprised of one or more macromers. The hydrogel or its macromers can comprise components including, but not limited to, a polymer backbone, stimuli responsive molecules as pendants or within the chain, photolabile moieties, end groups, and/or cross-linking agents. The polymer can be formed by way of the macromers cross-linking or undergoing a covalently bonded reaction such as “Click” reaction. In one aspect, one or more of the macromers can contain one or more photolabile moieties within the backbone or as pendant groups which can be degraded upon exposure to light such as ultraviolet or infrared light. The photolabile molecule can be synthetically incorporated into the macromer through a linkage to a heteroatom i.e. oxygen, sulfur, or nitrogen as an ether, thioether, ester, or amine. The structure of the photolabile moiety as well as the atom to which it is attached may affect the efficiency and wavelength required for photodegradation. Examples of photolabile moieties include the nitrobenzyl ether-derived moiety described by A. Kloxin (see A. Kloxin et al., “Photodegradable hydrogels for dynamic tuning of physical and chemical properties”, Science. 2009 Apr. 3; 324(5923): 59-63 and U.S. Pat. No. 8,343,710, incorporated by reference in its entirety), as well as those described in U.S. Pat. No. 9,180,196, U.S. Patent Application Publication Nos. US 20160153999 and 20120149781A1, and International Patent Application Publication No. WO2015168090A1, incorporated by reference herein in their entireties. Thus, upon exposure to a light stimulus, the occlusive implant can degrade or depolymerize as a result of degradation of the photolabile moiety.

The implant, such as a polymeric occlusive device or hydrogel, can comprise up-converting particles (UCNPs). UCNPs can convert low-energy and deeply penetrating NIR to high-energy radiation, such as UV/visible/NIR spectral range through a phenomenon known as photon upconversion. Monotonic UCNPs can be synthesized in a controlled fashion with lanthanide (Ln 3+) in the host lattice. Other sensitizers such as trivalent Yb 3+ and Nd 3+ ions can be activated by 980 nm and 800 nm light. Once activated, the conversion from NIR to UV light can cleave the photolabile moieties within the device to cause degradation, dissolution, disintegration, or reversal. In one embodiment, the apparatus of the invention releases a stimulus of NIR energy to activate the UCNPs.

Existing light-emitting medical devices, such as the ones used for lithotripsy or tissue ablation, primarily utilize long wavelength EMR and generate substantial heat. To the best of the knowledge of the present inventors, there are no devices that can administer specific stimuli to a stimuli-responsive implant, such as for dissolving or reversing it, while avoiding tissue damage.

In embodiments, the apparatus of the invention includes components such as a power source, a user interface, a catheter and/or needle, a fiber-coupled light source, a camera, and/or an irrigation system, in a combination operable to remove an implant. In one embodiment, the apparatus of invention includes an assembly which includes, but is not limited to, optical fibers, mechanical holding and mounting hardware, and fused silica capillaries. The assembly can vary with respect to the fiber or capillary type, fiber size (e.g. core, clad, buffer), overall assembly size, termination type (e.g. SMA, ST, shaped), end finish, numerical aperture of fiber, shaped end-tips, insertion loss, fiber anchoring (e.g. epoxy, crimp), a jacket, and bend diameters.

In one embodiment, power is supplied to the device via 60V or 120V AC current. However, embodiments of the device can be compatible with other voltages according to the single-phase voltage standard that is used in particular countries or regions. In general, this can be in the range of 100-127 volts or 220-240 volts. A representative list of single-phase voltage standard by country can be found on the internet at the world standards website (see http://www.worldstandards.eu/electricity/plug-voltage-by-country/). In one embodiment, the power is supplied to the device by a removable, rechargeable battery pack. In one embodiment, the device can be charged using a charging dock. In one embodiment, the power is tunable.

In one embodiment, the user interface for the device includes a mechanism to advance and retract the stimuli introducing catheter. The user interface can include a dial, switch, or programmable interface that allows for modification of the magnitude or type of stimulus introduced. In one embodiment, this can include modification of the EMR intensity, including modification of the intensity, the Boolean state of the signal, the frequency of the pulses of the signal, and/or the modification of the wavelength of the EMR. In another embodiment, the user interface can control the camera. In another embodiment, the user interface can allow for control of a flushing solution, including the Boolean state of the flush, the fluid flow rate of the flush, and/or the type of solution being flushed.

In one embodiment, the hand-held catheterization device includes a miniature camera at the tip of the device such as a fiber optic endoscope or fiberscope. The fiberscope, in conjunction with light emitted from the catheterization device, provides capabilities for visualization of the occlusion in situ on a display of the user interface. In this embodiment, the catheterization device can be advanced through the lumen until video on the display confirms that the device has reached the implant. Further, the fiberscope can confirm removal of the implant after one or more stimuli are provided through the catheterization device.

In another embodiment, the apparatus includes miniaturized tools such as drills, boring devices, rotating blades, lances, vibrating hammers, nanobot, or any other tool capable of delivering a mechanical stimulus tethered to the end of the device that is capable of physically removing portions of the occlusive device and/or breaking up the occlusion through mechanical stimuli. The device can have one or more tools which may be capable of grinding, sawing, piercing, boring, and/or drilling through the occlusion. The one or more tools can be controllable by way of the user interface. In another embodiment, the apparatus includes a basket or any other tool capable of capturing pieces or remains of the implant.

In one embodiment, the user interface includes a computing device or instrument that includes a processor (CPU), graphics processing unit (GPU), and non-transitory computer readable storage media such as RAM and a conventional hard drive, as well as a display. Other components of the computing device can include a database stored on the non-transitory computer readable storage media. As used in the context of this specification, a non-transitory computer-readable medium (or media) can include any kind of computer memory, including magnetic storage media, optical storage media, nonvolatile memory storage media, and volatile memory. Non-limiting examples of non-transitory computer-readable storage media include floppy disks, magnetic tape, conventional hard disks, CD-ROM, DVD-ROM, BLU-RAY, Flash ROM, memory cards, optical drives, solid state drives, flash drives, erasable programmable read only memory (EPROM), electrically erasable programmable read-only memory (EEPROM), non-volatile ROM, and RAM. The non-transitory computer readable media can include a set of computer-executable instructions, or software for implementing the methods, processes, operations, and algorithms of the invention. The computer-readable instructions can be programmed in any suitable programming language, including JavaScript, C, C#, C++, Java, Python, Perl, Ruby, Swift, Visual Basic, and Objective C.

In one embodiment, the apparatus includes a catheter or needle or combination of both by which external stimuli can be introduced. The external stimulus can be introduced and the needle and/or catheter can be configured to deliver one or more stimulus subdermally, percutaneously, or intraluminally, to reverse the implant. The apparatus can include a needle-sheathed catheter or a catheter-sheathed needle. The maximum needle size/gauge is determined by the lumen of the vessel, duct, or organ which will receive the external stimulus and as a result the exact size of catheter, needle, or instrument is not critical so long as it is shaped and sized appropriately for a particular application. The gauged needle and/or catheter can have a diameter ranging for example between 100 um and 5 mm, including 0.1 mm, 0.2 mm, 0.3 mm, 0.4 mm, 0.5 mm, 0.6 mm, 0.7 mm, 0.8 mm, 0.9 mm, 1.0 mm, 1 mm, 2 mm, 3 mm, 4 mm, or 5 mm. In one aspect, the needle diameter is preferably between 0.3 mm to 1 mm. In embodiments, the size of the needle and/or catheter can be from 6 gauge to 34 gauge, such as from 10 gauge to 34 gauge, or from5 15 gauge to 32 gauge, or from 20 gauge to 26 gauge, or from 22 gauge to 26 gauge, and so on. In other embodiments, the size of the needle is between 21 gauge and 31 gauge. In other embodiments, the needle can be extra extra thin walled (XXTW), extra thin walled (XTW), thin walled (TW), or regular walled (RW). Standard needle sizes are readily available such as at http://www.sigmaaldrich.com/chemistry/stockroom-reagents/learning-center/technical-library/needle-gauge-chart.html. In one embodiment, the needle is used to introduce a secondary catheter within the lumen of the vessel. In one aspect, the needle or catheter can have a length between 0.1 inch and 15 inches, preferably from 0.5 inch to 10 inches, such as from 0.8 to 5 inches, or from 0.4 to 1 or 2 or 3 inches. In one aspect, the needle is echogenic, or visible on ultrasound.

The needle or catheter system can include a single lumen. In one embodiment, the needle or catheter remains within the body system during stimulus exposure. In another embodiment, the needle or catheter is removed from the body cavity after being utilized to introduce the stimuli within the body. In one embodiment, the needle or catheter system contains multiple lumen, which can be utilized to introduce multiple stimuli to the implant simultaneously or in a particular sequence. In another embodiment, the needle or catheter acts as a space holder to allow for the introduction of a secondary stimuli-introducing mechanism.

In one embodiment, the needle acts to introduce a multi-lumen catheter to the body system. In one embodiment, such a multi-lumen catheter includes a single tubular system with multiple lumen running parallel to each other. In another embodiment, such a multi-lumen catheter includes a nested series of catheters, in that the sheath and lumen of one tubular structure sits internal to another. Each lumen of catheter can include the same or a separate system for delivering a unique stimulus to the occlusive implantation. For example, each lumen of the multilumen catheter can include a fiber optic system, an irrigation system, a fiberscope, or a miniature ultrasound probe. For example, the Olympus UM-2R, 12 MHz ultrasound probe, and UM-3R, 20 MHz probe, have an outer diameter of just 2.5 mm (Olympus America Inc., Center Valley, Pa.).

In one embodiment, the stimuli introducing component of the device includes a fiber optic in isolation or multiple fiber optics or in combination with any other stimuli.

In one embodiment, EMR is introduced within the body-system by way of a fiber optic catheter. In such an embodiment, the fiber optic catheter is coupled to an LED or other light source such as a laser that remains external to the body system, contained within the device. The fiber optic catheter is then introduced to the interior of the body system via minimally invasive methods, allowing illumination of an implant.

In one embodiment, the fiber optic is advanced within the body-system by a secondary mechanical system or actuator. The fiber optic catheter can be advanced and retracted by rotary motion, unpowered linear action, or powered rotary or powered linear action. The fiber optic catheter can be advanced and retracted according to commands introduced at the user interface of the device.

In one embodiment, the fiber catheter includes layers of materials with varying light-refracting properties. For example, the interior can include high —OH silica, while the sheathing can include low —OH silica. In embodiments, the silica is doped with materials to raise the refractive index (e.g. with GeO2 or Al2O3) or to lower it (e.g. with fluorine or B2O3). (see https://www.rp-photonics.com/silica_fibers.html).

In one embodiment, the light emitting end of the fiber optic has a variety of sculpted tips that create different illumination patterns including, but not limited to, “up” taper, “down” taper, lens (convex), lens (concave), lens (spherical ball), diffuser, side-fire, ring-of-light, and angled end. The fiber sculpted tip can be chosen based on the application and type of implantation that requires exposure. The illumination pattern can have a shape or configuration that can be linear, circular, rectilinear, curvilinear, sideways, or can increase/decrease light divergence. In embodiments, the devices can be configured to emit circular or arced illumination patterns from 0-360 degrees or any range in between including from 15-90 degrees, 30-180 degrees, 60-120 degrees, 90-240 degrees, 180-300 degrees, 45 to 150 degrees, and soon.

In one embodiment, collimation or coupling components are used to provide a stable platform for coupling light into and out of FC/PC, FC/APC, SMA, LC/PC, SC/PC, and/or ST/PC terminated fibers. The collimation or coupling component can be fixed or adjustable. The collimation or coupling component directs a laser beam from the end of the fiber while maintaining diffraction-limited performance at the desired wavelength.

In one embodiment, the fiber-coupled LED includes a single LED that is coupled to the optical fiber using the butt-coupling technique. The optical fiber can have a diameter that can be between 1 and 1000 microns, or more preferably between 200 and 500 microns, such as from 1 micron to 750 microns, or from 10 microns to 350 microns, or from 50 microns to 150 microns, or from 100 microns to 480 microns, and so on. The optical fiber can also have a diameter in the millimeter range, such as from 1-10 mm, 1-8 mm, 1-5 mm, 2-4 mm, or 2-3 mm for example for arterial or ductal applications. One of skill in the art will know how to upsize or downsize the instrumentation for a particular application.

In one embodiment, two or more, such as more than two, optical fibers can be used. The bundle of optical fibers can be used to increase the light intensity or introduce multiple wavelengths. The bundle of optical fibers can have a total diameter between 1000 microns to 10 mm. For instance, for an artery, the total optical fiber or fiber bundle diameter can be between 1 mm to 2 mm for a penile artery, 3 mm to 4 mm for a coronary artery, 5 mm to 7 mm for a carotid artery, and 6 mm to 8 mm for a femoral artery. Similarly, the total optical fiber bundle diameter can be between 2 mm to 4 mm for a hepatic duct, and 1 mm to 3 mm for a pancreatic duct. Thus, the total optical fiber or fiber bundle diameter can be adjusted according to the particular clinical application (e.g., the target vessel in which one wishes to remove an occlusion). In one aspect, each fiber optic can be run through a different lumen of the catheter or needle system. In another aspect, the fiber optics are joined or fused together to run in parallel through a single lumen. In another aspect, bundles fibers can run in parallel in one or more lumens of a multi-lumen catheter.

The coupling efficiency can be dependent on the core diameter and numerical aperture of the connected fiber. The LED can be mounted to a heat sink. A high-powered LED properly mounted to a heat sink exhibits better thermal management over time than an LED without a heat sink. The LED can emit light in the following colors: red, green, blue, amber, violet, warm white, cool white, ultra-violet. The LED can be mounted to printed circuit boards using surface-mount technology (SMT), also known as a surface-mounted device (SMD).

The LED can be high-power and high-current. The LED can also comprise a low or high thermal resistant material. For high-power, high-current LEDs, a low thermal resistant material is preferred. The forward voltage (V) of the LED can be from 0 to 5 V, such as from 0 to 1 V, 1 V-2 V, 2 V-3V, 3V-4V, or 4V-5V. The forward current (I_{F}) of the LED can be from 0 to 2,000 mA, such as from 200 to 400 mA, 400 to 600 mA, 600 to 800 mA, 800 to 1,000 mA, 1,000 to 1,200 mA, 1,200 to 1,400 mA, 1,400 to 1,600 mA, 1,600 to 1,800 mA, and 1,800 to 2,000 mA. The modulation frequency of the LED can be in the range of 1000 Hz and 3000 Hz, including 1100 Hz, 1200 Hz, 1300 Hz, 1400 Hz, 1500 Hz, 1600 Hz, 1700 Hz, 1800 Hz, 1900 Hz, 2000 Hz, 2100 Hz, 2200 Hz, 2300 Hz, 2400 Hz, 2500 Hz, 2600 Hz, 2700 Hz, 2800 Hz, 2900 Hz, or within any range encompassing any of these values such as from 1,600 Hz to 2400 Hz, 1400 Hz to 2500 Hz, 1700 Hz to 2300 Hz, 1100 Hz to 1900 Hz, 1400 Hz to 1600 Hz, 2300 Hz to 2600 Hz, and so on. The modulation shape of the LED can be varied as well such as triangle, single, or square.

Light emitting diodes have a divergent light emission, with radiance degrading from the center of the cone of irradiation. Optical fiber exhibits a narrow angle of acceptance, predicted as falling between twelve and twenty degrees to normal. Efficiency of the coupling then can be greatly improved by including a lensing system between the fiber optic and the LED.

In one embodiment, the fiber coupled LED involves a system of lensing to increase the coupling efficiency of the system. Such as system can include a microlens, a larger optical lens, or any combinatorial lensing system to more efficiently target the LEDs radiant energy to the fiber acceptance cone.

In one embodiment, the apparatus emits short wavelength electromagnetic radiation. The wavelength can range from 10⁻⁶ nm (gamma) to 2,500 nm (deep violet). The wavelength can range from 365 nm to 405 nm, or from 405 nm to 1000 nm, or from 200 nm to 2,500 nm, or from 250 nm to 450 nm, or from 300 nm to 425 nm, or from 330 nm to 420 nm, or from 350 nm to 390 nm, or from 365 nm to 405 nm, or from 330 and 460 nm, or from 370 nm to 440 nm, or from 405 nm to 500 nm, or from 500 nm to 800 nm, or from 700 nm to 2,500 nm or from 1000 nm to 10⁵ m. The wavelength emitted can depend on the implantation and the wavelength required for the implantation to be stimulated. For example, the implant can be modified using wavelengths between 300 nm and 500 nm, such as from 300 nm to 450 nm, or from 200 nm to 410 nm, or from 250 nm to 350 nm, or from 320 nm to 380 nm, or from 280 nm to 405 nm, or more preferably, between 365 nm and 405 nm, or at any range recited herein.

In embodiments, the apparatus includes a UV lamp coupled with the optical fiber. The UV lamp can emit light in UV-A, UV-B, or UV-C bands. In other embodiments, the apparatus includes an infrared lamp coupled with the optical fiber. In other embodiments, the apparatus includes a visible lamp or LED coupled with the optical fiber. In other embodiments, the apparatus includes a laser coupled with the optical fiber. The laser can be chosen to emit a wavelength from ultraviolet to visible to infrared. Non-limiting categories of laser sources include solid-state lasers, gas lasers, excimer lasers, dye lasers, and semiconductor lasers. An excimer laser is a non-limiting example of a laser emitting at ultraviolet frequencies, while a CO2 laser is a non-limiting example of a laser emitting at infrared frequencies. The choice of the laser will depend on the particular wavelength of light emitted and its relative absorption by the occlusive device. In one embodiment, the laser is a tunable laser which allows adjustment of the output wavelength. Descriptions of various laser sources are available in the art including Thyagarajan, K., Ghatak, Ajoy, Lasers: Fundamentals and Applications, Springer US, 2011, ISBN-13:9781441964410, incorporated by reference herein, as well as The Encyclopedia of Laser Physics and Technology (available online at https://www.rp-photonics.com/encyclopedia.html).

Various other sources of EMR wavelengths are known. For example, for gamma rays, radioactive sources such as ¹⁹²Ir, ⁶⁰Co or ¹³⁷Cs are used. For X-rays, an X-ray source such as an X-ray tube is used in conjunction with a collimator and a filter.

Additionally, the device can include a probe that emits radiofrequency waves or microwaves, which are converted to heat in situ. For example, the device can include a miniature radiofrequency probe. The probe emits radiofrequency radiation which results in both resistive and conductive heating of tissue in contact with the probe. In embodiments of methods of the invention, the probe can contact the occlusion itself, which can result in resistive and conductive heating of the occlusion. Alternatively, or in addition, the device can include a miniaturized tip that heats through electrical resistance to provide thermal energy to the occlusion. In embodiments, the needle and/or catheter can provide for cooling. In other embodiments, the miniaturized tip is configured to vibrate at selected frequencies. The occlusion can be chemically formulated such that it dissolves upon heating or vibrational energy.

In one embodiment, the apparatus is capable of introducing a particular energy level of EMR to the implantation. The light intensity can range from 0.1-40 J/cm² such as from 0.1-1 J/cm², 1-5 J/cm², 5-10 J/cm², 10-15 J/cm², 15-20 J/cm², 20-25 J/cm², 25-30 J/cm², 30-35 J/cm², or 35-40 J/cm². It is preferred that less than 5.25 J/cm² of light intensity is used for in vivo applications, given that light intensities higher than 5.25 J/cm² have been reported to be toxic to human mesenchymal stem cells according to Wong et. al. (see Wong et al., “Low-Dose, Long-Wave UV Light Does Not Affect Gene Expression of Human Mesenchymal Stem Cells”, PLOS ONE, Sep. 29, 2015 (https://doi.org/10.1371/journal.pone.0139307).

The light intensity can be flood based (non-polarized light) or laser (polarized). Polarized laser light can allow for increased degradation with lower light intensity due to tuning of the wavelength to the specific frequency that interacts with the implant. Furthermore, lowered light intensity can contribute to a lower degree of potentially adverse cellular effects. The EMR such as UV light can be collimated or can be partially shielded with an opaque photomask to create exposure gradients. The photomask can be moved at various rates including 0.5, 1.2, 2.4 mm/min. Further, the frequency of the light stimulus can be varied. For example, ultraviolet light has frequencies that range from 8×10¹⁴ Hz to 3×10¹⁶ Hz. If infrared light is used, the frequency can range from 300 GHz to 450 THz. The light stimulus can also be provided in pulses.

In one embodiment, methods of the invention include introducing the needle or catheter into the lumen of a bodily duct, vessel, tissue, interstitial space, or organ containing the implantation. The vessel can first be punctured using a hypodermic needle and then a single lumen catheter or multi-lumen catheter can be inserted into the area of the implanted device, such as for example into, onto, near, or surrounding the occlusive device or implant. An over-the-needle catheter can also be used. Then, a stimulus such as EMR can be introduced through the catheter or needle. For example, the light-conducting fiber can be introduced through the catheter or needle such that the fiber optic is able to be extended into the lumen of the vessel or cavity containing the implantation and is able to apply light onto the surface of the implantation, the side of the implantation, or is able to penetrate the implantation to apply light from within. The methods can include touching the implantation or not when delivering light. In one embodiment, the needle and/or light-conducting fiber punctures or enters the composition then delivers light, such as delivering 360 degrees of light (around the needle or catheter) within the lumen to treat the composition disposed therein. This is especially useful for implantations that are soft materials, such as hydrogels. The illumination pattern can be varied to treat only part of the occlusive device and/or to administer light/energy from only part of the needle or catheter. For example, the device can comprise an adjustable sheath or other structure for blocking or insulating the light/energy in a manner such that light/energy can be emitted from the device and/or administered to an occlusive device from 5-180 degrees, or from 10-165 degrees, or from 20-135 degrees, or from 30-110 degrees, or from 45-150 degrees, or from 50-95 degrees, or from 55-85 degrees, or from 75-120 degrees, or from 60-110 degrees, and so on, or any range of amount disclosed herein, around an axis running lengthwise through the needle/catheter.

In one embodiment, the exposure time of the stimulus can be seconds, minutes, or hours, but is preferably from 1 second to 60 minutes. The implantation can be removed, impacted, or reversed by the apparatus within seconds, minutes, or hours. In embodiments, the amount of time sufficient to degrade a particular polymer occlusion will depend on the particular polymer composition, degradation protocol, stimuli that are used, and time such as from 10 seconds to 1 minute, up to 2 minutes, or up to 3 minutes, or up to 4 minutes, or up to 5 minutes, or up to 6 minutes, or up to 7 minutes, or up to 8 minutes, or up to 9 minutes, up to 10 minutes, up to 20 minutes, up to 30 minutes, up to 60 minutes, up to 1 hour, up to 2 hours, up to 5 hours, up to 10 hours, or up to 12 hours, or longer. The use of multiple stimuli and/or higher intensity for degradation can result in shorter exposure times that are effective in degrading the polymer occlusion. In one embodiment, exposure takes place over the course of one or multiple clinical visits, with each exposure further degrading the implanted polymer. This is especially useful for drug-delivery applications that require on-command delivery of the drug from the implant and may require multiple activations. The stimulus or stimuli can also be used to increase or reduce the implant's swelling, mesh/pore size, charge, and/or solubility. The time exposure can also be performed over the course of multiple treatments for the same or varying amounts of time. For example, the stimulus can be applied once or more per selected time period, such as per second, minute, hour, day, week or year. For example, the treatment can be applied for a selected amount of time at a selected interval from the time periods and intervals provided above, or for any amount of time or time period or combination thereof.

In one embodiment, the apparatus can be configured to introduce a fluid that is capable of acting on the implantation. The administered fluid can be capable of changing the charge or pH of the environment which the implantation is situated and/or reverse, dissolve, dislodge, or de-precipitate the implantation or assist in removing the reversed, dislodged, dissolved, or de-precipitated implant from the body. In embodiments, the fluid is capable of deteriorating, breaking down, degrading, disintegrating, reversing, dissolving, destroying, removing, dislodging, de-precipitating, liquefying, flushing and/or reducing, in whole or part, the occlusive implantation.

The fluid can be saline, phosphate-buffered saline, Ringer's solution, or a buffered solution, or any other non-toxic solutions or solvents. The fluid can be pressurized. The fluid can contain various buffering agents including citrate, phosphate, acetate, or carbonate for maintaining the pH of the solution. For example, the solutions can include sodium or potassium bicarbonate for maintaining a basic pH. In one aspect, the solution has a pH from 7.01-10. In another aspect, the fluid has a pH of 7 (neutral). In another aspect, the fluid has a pH from 4-6.99. According to embodiments, the occlusive implantation can be sensitive to changes in pH such that acidic and/or basic stimuli result in depolymerization of the implant. Further, the fluid can contain one or more agents (chemical or biological, as described below) to act on the implantation and result in dissolution or depolymerization. In addition, the fluid can be or include various organic solvents such as DMSO, or other organic solvents, that are capable of dissolving the polymer of the occlusive implant.

Included in embodiments of the irrigation system is a fluid source such as an IV bag of saline or another solution, an infusion pump such as a Harvard pump capable of being programmed to deliver the fluid through the catheter at a specific rate, and medical tubing such as polyethylene tubing connected to the irrigation system in the catheter. The infusion pump can be programmed to deliver the solution through the catheter at a constant level or in pulses or bursts that exert physical pressure on the occlusion. However, the infusion pump can also be programmed to limit the volume of fluid so that the vessel, duct, or organ does not rupture during administration.

In one embodiment, the apparatus includes a multi-lumen catheter or needle such that two or more different stimuli can be introduced simultaneously. The stimuli can include, but are not limited to, electromagnetic radiation, chemical agent, biological agent (e.g. an enzyme) mechanical stimulus, or irrigation i.e. saline or another solution. For example, the chemical agent can be one that reverses a polymer synthesized by Click Chemistry (see David A. Fulton, “Synthesis: Click chemistry gets reversible” Nature Chemistry 8, 899-900 (2016)). The chemical agent can also be a reducing agent such as glutathione which can break disulfide cross-links in a hydrogel. The biological agent can be a protease such as papain, bromelain, actinidin, ficin, or zingibain that reverses the gel by digesting fusion proteins, amino acid sequences, or peptides (natural or synthetic) that are cross-linked to the hydrogel. The chemical or biological agent can be delivered in a solution. The stimuli can be delivered in any combination such that each individual stimulus is delivered through a separate lumen of the catheter.

In one embodiment, the apparatus includes a single handheld unit, in which all systems and subsystems are contained. In one embodiment, the apparatus includes a handheld unit in which all systems which come in contact with a patient are disposable. In such an embodiment, disposable components can include but are not limited to the piercing needle, a section of fiber optic catheter, and a threaded connection head.

In another embodiment, the apparatus includes a non-consumable part (handle) with a consumable catheter/needle. In another embodiment, the apparatus is completely consumable using a built-in battery. As used herein, “consumable” is intended to mean its commercial sense, i.e. intended to be used and replaced.

In another embodiment, the power supply and a portion of the user interface are contained within a table mounted box. Power can be then transmitted to the handheld portion of the apparatus, which can include a LED light source, further user interface components, and a coupling point to the disposable catheter/fiber head.

In any such embodiment of the apparatus, the apparatus includes a subsystem that allows for the introduction of a fluid flush through the stimuli introducing catheter system. A fluid reservoir can be contained within the device itself, or the system can include a port to allow for the introduction of a fluid flush via a secondary syringe introduction system.

In one embodiment, the apparatus includes a disposable system, with all subsystems being contained in one handheld package.

In one embodiment, the apparatus includes a mechanical system, chemical system, and/or electromagnetic system to remove an implantation. The apparatus can include any number of types of systems or combination of systems to remove an implantation from the body by causing a physical or chemical effect on an implantation. In embodiments, methods for removal of the implantation are guided by ultrasound. In particular, ultrasound can be used to guide placement of the needle or catheter to the implant. For example, ultrasound can be used to identify an occlusive implant in the lumen of the vessel, such as a vas deferens or fallopian tube, as well as image a needle that can be used to introduce a catheter into the vessel. Further, the implantation can be imaged using a medical imaging modality prior to using the apparatus, such as ultrasound, MM, CT, x-ray, PET, PET-CT, or any combination thereof. The imaging can be used to determine the location, occlusive nature, length of the implant, or a combination thereof.

Alternatively or in addition, ultrasound can be used to assist in removing the occlusive implantation. For example, in one embodiment, focused ultrasound is applied at a particular frequency which causes microbubbles within the occlusive implantation to vibrate. At a particular threshold of intensity and/or frequency, the microbubbles can be destroyed, which can cause a local shock wave, resulting in cavitation and lysing of the gel. Thus, the use of ultrasound can provide a non-invasive method of reversing the occlusion. Accordingly, one embodiment of the invention provides a method of reversal of an occlusive implantation comprising applying ultrasonic energy to the occlusion at a frequency and/or intensity that is capable of destroying microbubbles inside the occlusion, thereby lysing and destroying the occlusion.

In one embodiment, a level of ultrasonic energy needed for microbubble cavitation is determined. For example, a detector transducer receives a scattered level of ultrasonic energy, indicative of stable cavitation. Accordingly, a method for in vitro or ex vivo testing of microbubble cavitation is used to determine acoustic pressures necessary for reversal. Once a measurement is recorded which is expected to adequately reverse, de-precipitate, liquefy, dissolve, or flush out the polymer gel, such a frequency can be used to reverse, de-precipitate, liquefy, dissolve, or flush out the polymer occlusion in a subject.

Various frequencies can be used for imaging the implant including contrast-pulse sequencing mode (7 MHZ), B-Mode imaging (14 MHZ), and frequencies in between. Other possible ultrasound modes that can be used for the inventive methods include 2D mode, fusion, harmonic imaging (THI), color mode or color power angio, CW doppler mode, PW doppler mode, M-Mode, anatomical M-mode (live or frozen image), B-Mode, color tissue doppler, PW tissue doppler, panoramic imaging, 3D/4D imaging, and dual imaging. In some embodiments, the frequencies used for imaging and/or reversing the implant are between 1 Hz and 20 MHZ, including 1, 2, 3, 4, 5, 6, 7, 8, 9, 10, 11, 12, 13, 14, 15, 16, 17, 18, 19, or 20 Hz, decahertz, kilohertz, or MHZ, or within any range encompassing any of these values such as 1-5 Hz, decahertz, kilohertz, or MHZ, 2-8 Hz, decahertz, kilohertz, or MHZ, 3-11 Hz, decahertz, kilohertz, or HZ, 5-14 Hz, decahertz, kilohertz, or MHZ, 11 to 19 HZ, decahertz, kilohertz, MHZ, and so on. Additionally, the ultrasound can be delivered at different intensities, such as between 0.1 to 1 W/cm², including 0.1, 0.2, 0.3, 0.4, 0.5, 0.6, 0.7, 0.8, 0.9, and 1.0 W/cm², or within any range encompassing any of these values such as 0.1 to 0.3 W/cm², 0.2 to 0.5 W/cm² 0.4 to 0.8 W/cm², 0.5 to 0.7 W/cm², 0.3 to 0.6 W/cm², and so on. Additionally, the ultrasonic energy can be delivered at a specific power, such as 0 to 20 Watts of energy, including 0, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20 Watts or within any range encompassing any of these values such as 0.2 to 1 Watts, 0.1 to 0.5 Watts, 2 to 6 Watts, 6 to 13 Watts, 9 to 20 Watts, and so on. Additionally, the ultrasonic energy can be delivered in pulsed or continuous mode. The ultrasound can be delivered through an ultrasound unit. The ultrasound unit can be portable. An example of a portable ultrasound unit for scrotal imaging is the LOGIQ V2, manufactured by GE Healthcare (Little Chalfont, United Kingdom). Another example of an ultrasound unit for scrotal imaging is the ClearVue 350 by Philips (Amsterdam, Netherlands).

In regards to safety, the FDA advises that the mechanical index (MI) and thermal index (TI) are kept below 1.90 and 6 degrees C., respectively.

According to embodiments, various ultrasound probes or transducers can be used for ultrasound imaging the vessel such as a vas deferens, including sector (phased array), linear and convex transducers. Ultrasound probes and their selection have been discussed in the literature (see T. L. Szabo et al., “Ultrasound Transducer Selection in Clinical Imaging Practice”, Journal of Ultrasound in Medicine, 2013, 32(4):573-582). Ultrasound transducers differ according to their piezoelectric crystal arrangement, physical dimensions, shape, footprint (aperture), and operating frequency. It is within the ability of a skilled artisan (e.g. urologist or ultrasound technician) to choose a transducer with appropriate characteristics to image the area of the vas deferens that has been isolated. A hand-held probe can be chosen for imaging that is small enough to image the vas without interfering with other aspects of the procedure such as administration or reversal of the implant.

This disclosure reports that ultrasound is the ideal imaging modality for performing or assisting with administering a stimulus to an implant, such as a polymer occlusion in the vas deferens. The relatively shallow depth at which the vas deferens sits allows for easy identification by a medium or high frequency ultrasound. Ultrasound is rarely used in clinical applications for imaging the vas deferens. Thus, prior to the present disclosure, methods for optimal imaging of the vas deferens were limited. To the best of the knowledge of the present inventors, ultrasound-guided, percutaneous administration of stimuli into the vas deferens has never been performed. Optionally using ultrasound as guidance for performing percutaneous procedures into the vas deferens is critically needed because: 1) every subject has different morphometric measurements of the vas (e.g. outer and inner diameter, depth, length), 2) the physician or other professional (e.g. technician, veterinarian, etc.) performing the procedure can visualize that the needle is inside the vas lumen as opposed to the smooth muscle layers of the vas, 3) the physician can visualize the implant and its morphometrics, 4) the physician can visualize the stimulus (e.g. fiber optic, solution, etc.) being administered into the lumen, 5) the physician knows that the stimulus administration was successful if the implant is degraded (e.g. smaller length) or no longer visible on the ultrasound.

An additional embodiment of the invention includes a method of reversal of an occlusion comprising non-surgically or surgically isolating the occluded vessel and administering a solvent or solution in the lumen of the vessel that is capable of dissolving the occlusion. For example, the method of reversal can rely on ultrasonic imaging to determine the location of the occlusion in the vessel. Then, the vessel such as a vas deferens can be isolated. Then, a solvent or solution which is capable of dissolving the occlusion can be administered into the lumen of the vessel. Alternatively, the solvent or solution can be used to “flush out” the occlusion. For example, the solvents can include DMSO and the solutions can include sodium or potassium bicarbonate. In one aspect, the solution has a pH from 8-9. As an alternative to bicarbonates, other alkaline solutions can be used. Anywhere from 0.01-20 cc of active agent, such as a solvent or solution, can be injected into the lumen of the vas deferens, such as from about 0.01 cc to 0.02 cc, 0.02 to 0.03 cc, 0.03 cc to 0.04 cc, 0.1 cc to 20 cc, 0.2 cc to 15 cc, 0.05 cc to 10 cc , 0.05 cc to 4 cc, or from 0.15 cc to 3 cc, 0.2 cc to 0.5 cc, 0.5 cc to 8 cc, and so on, or any range or amount based on these values. However, the rate and volume of injections are limited such that the injection force does not rupture the walls of the vessel. The dissolution of the polymer occlusion can then be monitored in real time using ultrasound. Absence of the occlusion and patency of the vessel lumen can be confirmed via ultrasound imaging. Further, in the case of removal of the occlusive device from the vas deferens, removal of the polymer occlusion can be confirmed through sperm counts.

The apparatus can be a handheld device with a screen similar to a cystoscope. The handheld device can be configured so that a user can push a button to release or extend the optical fiber. Alternatively, the apparatus can shine light above the skin to degrade the implant, such as an otoscope or dental curing device. This is especially useful for implants located in the subcutaneous space.

The apparatus of the invention has several applications or industrial uses, including male and female contraception and/or reversal thereof, occlusion of any organ, tissue, duct, etc. and/or reversal thereof; occlusion of artery to cause necrosis of tumor and/or reversal thereof; occlusion of aneurysm and/or reversal thereof; sustained release of factors, proteins, stem cells, drugs, antibodies, fertility boosting reagents, antibiotics, microbubbles, liposomes, or nanoparticles.

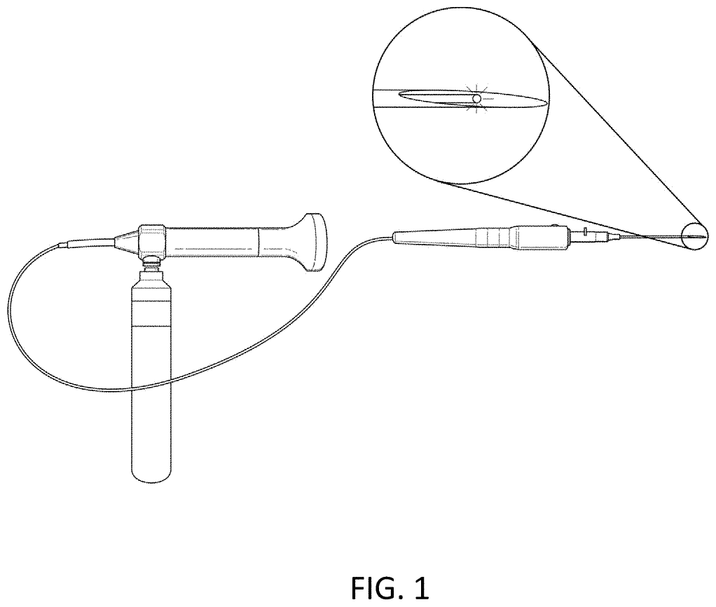

Turning now to the figures, FIG. 1 is a schematic diagram showing an embodiment of an apparatus or system of the invention. The apparatus or system includes a handheld device containing a power source and light source (e.g. LED). The light source is connected to a hand-held catheterization device by a fiber optic transmission cable. The hand-held catheterization device includes a light-emitting needle tip for applying a light stimulus to a polymer plug. The fiber optic can also have a sculpted tip. It is important to note that the dose amount and dose time of the stimulus as well as wavelength of the light applied can be tuned and controlled by altering the components of the system. Additionally, the method of introduction of the stimuli carrier can be performed with devices other than needles; including but not limited to catheters, tubing, and multi-lumen tubing. Not shown, the power source can be battery-powered or electrically-powered.

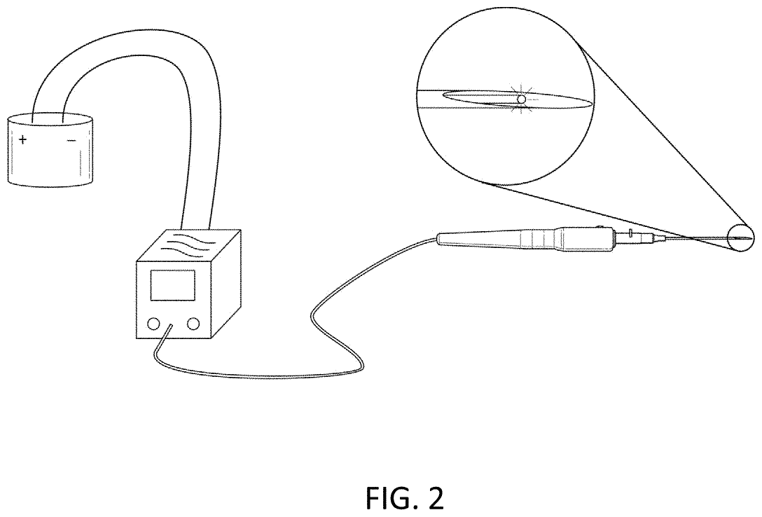

FIG. 2 is a schematic diagram showing an embodiment of an apparatus or system of the invention. The apparatus or system includes a power source that is connected to a light source (e.g. LED). The light source is connected to a hand-held catheterization device by a fiber optic transmission cable. The hand-held catheterization device includes a light-emitting needle tip for applying a light stimulus to a polymer plug. The fiber optic can also have a sculpted tip. It is important to note that the dose amount and dose time of the stimulus as well as wavelength of the light applied can be tuned and controlled by altering the components of the system. Additionally, the method of introduction of the stimuli carrier can be performed with devices other than needles; including but not limited to catheters, tubing, and multi-lumen tubing.

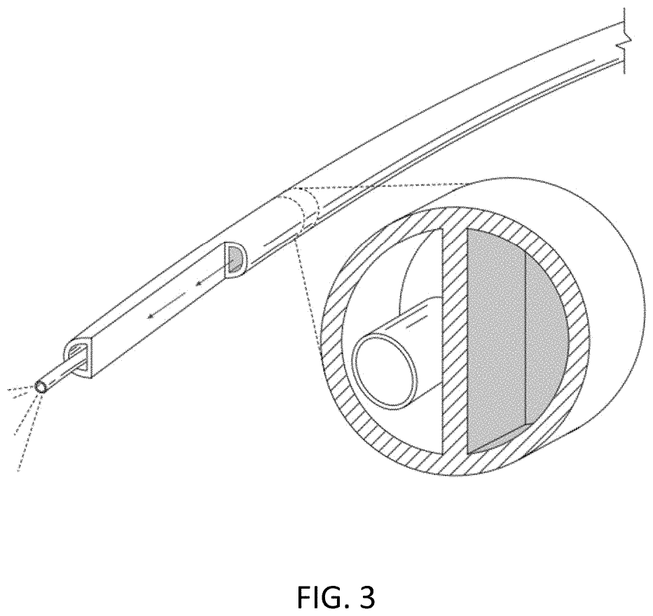

FIG. 3 is a schematic diagram showing a device according to embodiments of the invention, such as a catheter device as well as a cross-section of the device. The diagram shows a catheter with multiple lumens, such as two lumens (in this case formed by a wall bisecting the catheter), where one lumen allows passage of a stimulus-delivering device such a fiber optic or bundle of fiber optics and another lumen allows passage or delivery of a fluid stimulus such as an enzymatic solution, pH solution, or saline flush. It should be noted that multiple lumens can allow a combination of fiber optics with different wavelengths of light and/or 2 or more solutions can be delivered using the device.

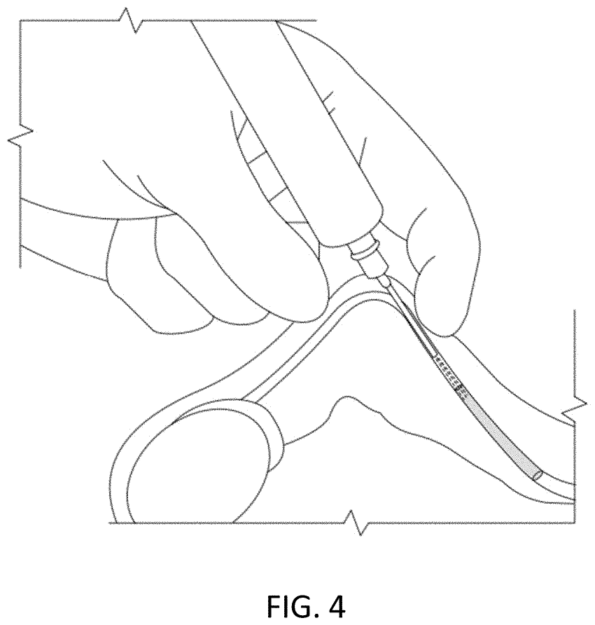

FIG. 4 is a schematic diagram showing an embodiment of a method in which a stimulus is delivered to an occlusion, such as in the lumen of the vas deferens, through a device according to the invention, such as a catheter device. According to embodiments, any stimulus according to those described herein can be delivered. Delivery of the stimulus has an effect on the occlusion to disintegrate, de-precipitate, dislodge, and/or dissolve the occlusion, thereby reversing or otherwise interfering with functionality of the occlusion and the contraception produced by the occlusion.

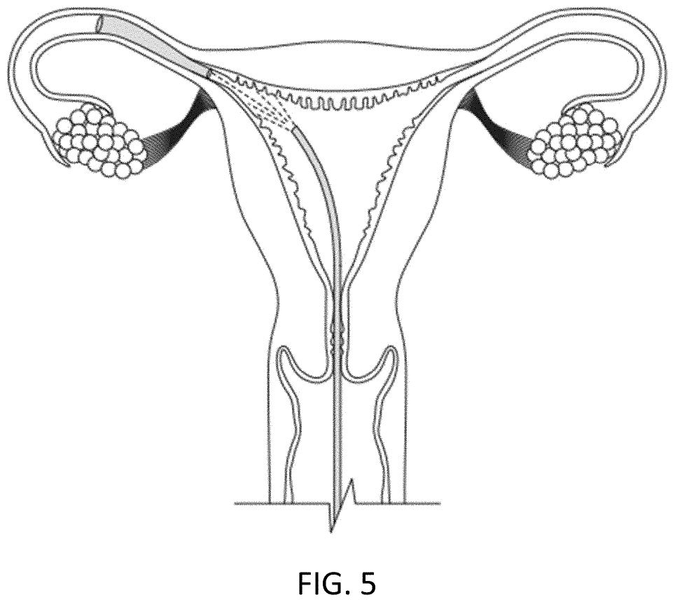

FIG. 5 is a schematic diagram showing an embodiment of a method in which a stimulus is delivered to an occlusion in the lumen of a body, such as an oviduct, through a device of the invention, such as a catheter device. According to embodiments, any stimulus according to those described herein can be delivered. Delivery of the stimulus has an effect on the occlusion to disintegrate, de-precipitate, dislodge, and/or dissolve the occlusion, thereby reversing or otherwise interfering with functionality of the occlusion and the contraception produced by the occlusion.

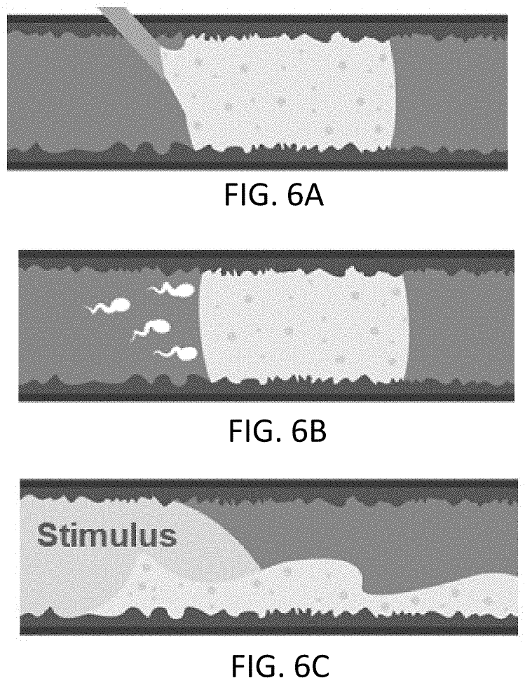

FIG. 6A is an illustration showing administration of an occlusive polymer into the lumen of a vessel such as a vas deferens according to one embodiment. The occlusive polymer is administered through a needle/catheter such that the tip of the needle penetrates the muscular wall of the vas deferens and is inserted into the lumen. A polymer or polymers are injected into the lumen of a vessel thus forming an occlusion designed to block molecules of a certain size. The lumen can be any bodily lumen or space, including a vessel such as an artery, vein, capillary, lymphatic vessel, a vas deferens; a duct such as a bile duct, a hepatic duct, a cystic duct, a pancreatic duct, or a parotid duct; a tube such a fallopian tube; an organ such as a uterus, or any organ of the gastrointestinal tract or respiratory system; an interstitial space; or the like.

FIG. 6B is an illustration showing the placed vas-occlusive polymer plug into the lumen of the vas deferens according to one embodiment. The vas-occlusive polymer plug physically blocks sperm cells from progressing through the vas deferens.

FIG. 6C is an illustration showing application of a stimulus (such as electromagnetic radiation, chemical agent, biological agent (e.g. an enzyme) mechanical stimulus, or irrigation) into the lumen of a vessel such as a vas deferens at the site of the polymer plug according to one embodiment. The stimulus has an effect on the polymer plug to reverse the occlusion such that in the case of the vas deferens, fertility is restored.

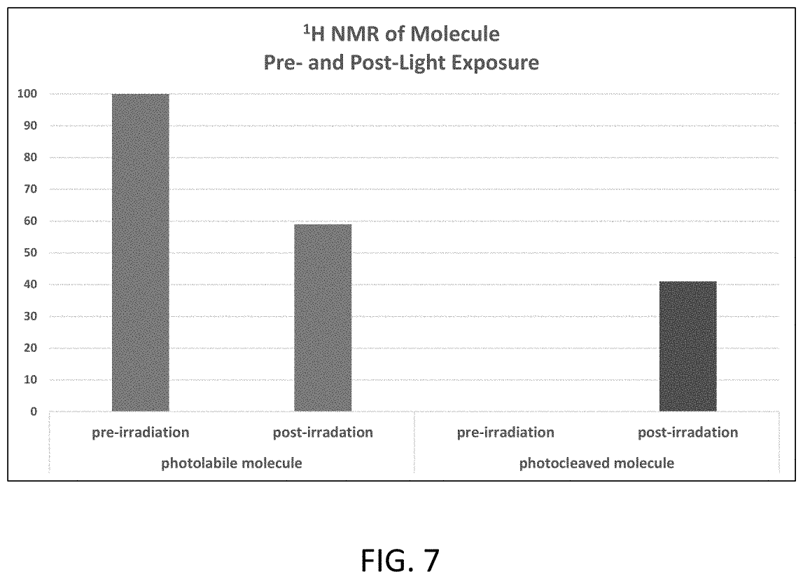

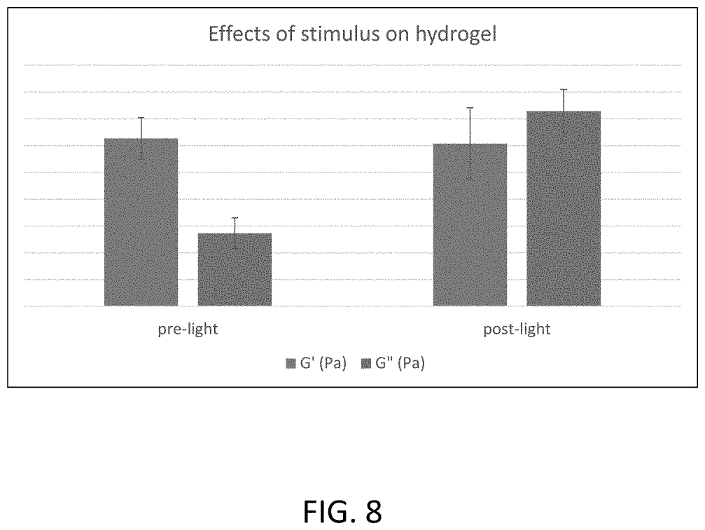

FIG. 7 is a bar graph which shows the chemical transformation of a photolabile molecule in solution after short exposure to UV light using the apparatus described herein. The extent of chemical transformation was 41% after the dose applied. It is important to note that the pre-irradiation bar for the photocleaved molecule is 0%. FIG. 7 along with FIG. 8 demonstrate that the extent of photoinduced chemical transformation of light-responsive molecules and hydrogels can be tuned based upon dose applied to the system and that this chemical transformation can be monitored by NMR and rheology. The degree of chemical transformation can be varied based on factors including the dose intensity, dose time, as well as wavelength of the light applied.

FIG. 8 is a bar graph demonstrating the change in the properties of an implant (in this example: a hydrogel) containing a stimuli-responsive component (in this example: a photolabile moiety in the polymer backbone). Before application of the stimulus, the implant is a soft, elastic material (pre-light portion of the graph) in which the G′ (storage modulus) is greater than the G″ (loss modulus). When the G′ is greater than the G″, the implant is considered a non-flowing material, meaning that when energy or force is applied, the material can store and dissipate the energy while still maintaining a shape/network. It can be envisioned that if this material is implanted into the lumen of a vessel, it would create an effective occlusion based on the properties as measured by parallel rheometry. Upon application of a stimulus (in this example: UV-A light), the properties of the implant change such that the G′ is now less than the G″ indicating that the sample is no longer a viscoelastic material. In this state, the material can no longer store applied energy and as such, it is able to flow. Flow can be further assisted with a second stimulus such as mechanical or solution (i.e. saline flush). Therefore, if this material were within a vessel, it would no longer be able to occlude that vessel.

The present invention has been described with reference to particular embodiments having various features. In light of the disclosure provided above, it will be apparent to those skilled in the art that various modifications and variations can be made in the practice of the present invention without departing from the scope or spirit of the invention. Any apparatus, system or device described herein may be used in any method described herein or any method otherwise available at any time. Likewise, any method described herein can be performed by any apparatus, device, or system described herein or otherwise available at any time. One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Other embodiments of the invention will be apparent to those skilled in the art from consideration of the specification and practice of the invention.

It is noted in particular that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all of the references cited in this disclosure are each individually incorporated by reference herein in their entireties and as such are intended to provide an efficient way of supplementing the enabling disclosure of this invention as well as provide background detailing the level of ordinary skill in the art.

1. A device for modifying an implant in a body, the device comprising:
one or more probes configured to administer one or more stimuli to an implant in a body;
one or more stimuli capable of modifying the implant structurally and/or chemically; and
a power source for enabling delivery of the one or more stimuli to the implant.

2. (canceled)

3. A device for delivering at least one stimulus to modify an occlusive implantation, the device comprising:
a power source;
a light source in operable connection with the power source; and
a catheterization device in operable connection with the light source by way of a fiber optic cable, the catheterization device comprising:
a needle or catheter;
a fiber optic disposed within the lumen of the needle or catheter capable of providing a stimulus which is light in the range of about 200 to 1,000 nm.

4. The device of claim 3, wherein: the needle or catheter comprising comprises at least two lumens;
the fiber optic is disposed within a first lumen of the needle or catheter; and
second lumen of the needle or catheter is capable of delivering a second stimulus.

5. The device of claim 1, wherein the probe comprises an irrigation system, and one or more of the stimuli is a fluid.

6. (canceled)

7. The device of claim 1, wherein one or more of the stimuli is light with a wavelength which ranges from 200 nm to 1,000 nm.

8. The device of claim 1, wherein one or more of the stimuli is light with an energy which ranges from 0.01-10 J/cm².

9. (canceled)

10. The device of claim 1, wherein the probe comprises a lumen capable of delivering one or more of the stimuli.

11. (canceled)

12. The device of claim 1, wherein the stimulus is ultraviolet, infrared, monochromatic, or visible light.

13-15. (canceled)

16. The device of claim 5, wherein the fluid comprises a chemical or biological agent capable of depolymerizing or dissolving the implant.

17-18. (canceled)

19. A method comprising:
providing a probe, needle or catheter configured to administer one or more stimuli to an implant in a body;
introducing the probe, needle or catheter into a body lumen having the implant disposed therein; and
applying one or more stimuli by way of the probe, needle or catheter to the implant and modifying the implant in situ.

20. The method of claim 19, wherein the implant is disposed in a vas deferens, fallopian tube, aneurysm, blood vessel, duct, tumor, tissue, interstitial tissue, or organ.

21. (canceled)

22. The method of claim 19, wherein the implant is a gel, hydrogel, mesh, film, composition, or device.

23. (canceled)

24. The method of claim 19, wherein the modifying comprises reversal, degradation, dissolution, and/or de-precipitation of the implant.

25. (canceled)

26. The method of claim 19, wherein the implant occludes a reproductive tract resulting in contraception, and the modifying removes the occlusion resulting in reversal of the contraception.

27. The method of claim 19, wherein reversal of the implant restores a flow of fluid, cells, and/or proteins within the body.

28. (canceled)

29. The method of claim 19, wherein the one or more stimuli is used to modify or alter the porosity of the implant or implantation.

30-33. (canceled)

34. The method of claim 19, wherein one or more steps of the method are guided by an imaging modality comprising ultrasound, x-ray, MM, or CT, or any combination of these.

35. (canceled)

36. The method of claim 19, wherein at least one of the one or more stimuli is an irrigation solution and at least one of the one or more stimuli is light.

37-49. (canceled)

50. The device of claim 3, wherein the catheterization device is disposable.

51-54. (canceled)

55. The device of claim 3, wherein:
the fiber optic has an outside diameter of 200 to 500 microns; and
the fiber optic is disposed within the needle and/or catheter in a manner to protract all or a portion of the length of the fiber optic out of the needle and/or catheter and to retract all or a portion of the length of the fiber optic into the needle and/or catheter; and
the device is configured to deliver the light stimulus with an adjustable illumination pattern at any angle or range of angles from 0 to 360 degrees.
